(19) United States
(12) Patent Application Publication
(10) Pub. No.: US 2025/0082420 A1
(43) Pub. Date: Mar. 13, 2025

(54) METHOD AND SYSTEM FOR ROBOT GUIDED NEEDLE PLACEMENT

(71) Applicant: EDDA TECHNOLOGY, INC., Princeton, NJ (US)

(72) Inventors: Yuanfeng Mao, Mt Laurel, NJ (US); Guo-Qing Wei, Plainsboro, NJ (US); Li Fan, Belle Mead, NJ (US); Xiaolan Zeng, Princeton, NJ (US); Jianzhong Qian, Princeton Junction, NJ (US)

(21) Appl. No.: 18/465,288

(22) Filed: Sep. 12, 2023

(51) Int. Cl. A61B 34/30; A61B 17/34

(52) U.S. Cl. CPC A61B 34/30; A61B 17/3403; A61B 2034/107; A61B 2034/2059

(57) ABSTRACT

Method, system, medium, and implementation for robot-guided instrument insertion. A preplanned path between a three-dimensional (3D) entry pose on skin of a patient to a 3D pose of a target is generated for a surgery with a target inside the patient. The preplanned path is used by a robot as guidance to insert a surgical instrument from the 3D entry pose to reach the 3D pose of the target. During the surgery, a next pose is determined based on a current pose of the surgical instrument and the preplanned path as well as spatial relationship to surrounding anatomical structures and is used to move the surgical instrument thereto. An updated current pose of the instrument is then obtained via tracking when the robot is advancing the surgical instrument to the next pose. The process repeats until the instrument reaches the target pose.

### BACKGROUND

### 1. Technical Field

The present teaching relates to computers. More specifically, the present teaching relates to signal processing.

### 2. Technical Background

With the advancement of different technologies, more and more tasks are now performed with the assistance of computers. Different industries have benefited from such technological advancement, including the medical industry. Different medical data acquisition techniques, such as computerized tomography (CT) or magnetic resonance imaging (MRI), large volume of image data capturing anatomical information of a patient may be readily obtained. With sophisticated data processing algorithms running on fast computers with tremendous storage capacities, such large volume of medical data may be processed to identify anatomical structures of interest (e.g., organs, bones, blood vessels, or abnormal nodule), obtaining measurements for each object of interest (e.g., dimension of a nodule growing in an organ), quantifying anatomical structures (e.g., dimension and shape of abnormal nodules), and constructing three-dimensional (3D) models of organs and associated anatomical structures.

Such information may be used for a wide variety of medical purposes, including assisting in diagnosis, in presurgical planning, as well as during-surgery to provide certain guidance. For example, presurgical planning may be based on constructed 3D models of target organs and surrounding anatomical structures to, e.g., planning a route for a surgical instrument such as a biopsy needle to travel from skin of a patient to the target organ. This is illustrated in FIG. 1, there may be a target object 100 and other surrounding anatomical structures such as 120-1 and 120-2 under the skin 110 of a patient. To reach target 100 (e.g., a malignant tumor) to perform either resection or biopsy or ablation, a surgical instrument 140 may need to be inserted into the patient's skin 110 and travel towards the object 100 without touching the nearby critical anatomical structures 120-1 and 120-2. During pre-surgical planning, a route 130 from an insertion point on skin 110 to the target object 100 may be planned so that the tip of the surgical instrument is to follow to reach the target object.

Although such a surgical route may be generated with precision prior to a surgery based on 3D models of different anatomical parts, during the surgery, because of the deformable nature of anatomical parts, features or spatial relationships of anatomical parts usually change, e.g., anatomical structures 120-1 and 120-2 may become much closer or even overlap with the preplanned route. Such deformation makes it necessary to dynamically adapt the route to the real time situation to maneuver the surgical instrument to approach a target organ without collision with other anatomical parts. In some situation, a target organ may not even be visible to a surgeon, also making it very challenging. For instance, in a laparoscopic procedure, a surgeon sees only what a laparoscopic camera captures in a limited view. Without a view of a wider surrounding or what is under the surface of visible anatomical structure, it can also make it difficult to figure out what is the next step.

Thus, there is a need for a solution that addresses the challenges discussed above.

### SUMMARY

The teachings disclosed herein relate to methods, systems, and programming for information management. More particularly, the present teaching relates to methods, systems, and programming related to hash table and storage management using the same.

In one example, a method, implemented on a machine having at least one processor, storage, and a communication platform capable of connecting to a network for robot-guided instrument insertion. A preplanned path between a three-dimensional (3D) entry pose on skin of a patient to a 3D pose of a target is generated for a surgery with a target inside the patient. The preplanned path as well as spatial relationship to surrounding anatomical structures are used by a robot as guidance to advance a surgical instrument from the 3D entry pose to reach the 3D pose of the target. During the surgery, a next pose is determined based on a current pose of the surgical instrument and the preplanned path and is used to move the surgical instrument thereto. An updated current pose of the instrument is then obtained via tracking when the robot is inserting the surgical instrument to the next pose. The process repeats until the instrument reaches the target pose.

In a different example, a system is disclosed for robot-guided instrument insertion that includes a next pose determiner, a robot-guided instrument insertion controller, and a current instrument pose determiner. The next pose determiner is provided for determining a next pose based on a preplanned path generated for a surgery on a patient with respect to a target inside the patient. The preplanned path is between a three-dimensional (3D) entry pose on skin of the patient to a 3D pose of the target and is used, together with detected spatial relationship to surrounding anatomical structures, by a robot to advance a surgical instrument from the 3D entry pose to reach the 3D pose of the target. Each next pose is determined based on a current pose of the surgical instrument and the preplanned path. The robot-guided instrument insertion controller is for controlling the robot to move the surgical instrument to reach the next pose. The current instrument pose determiner is provided for obtaining an updated current pose of the surgical instrument via tracking. The steps of determining, controlling, and obtaining may be repeated until the surgical instrument reaches the 3D pose of the target.

Other concepts relate to software for implementing the present teaching. A software product, in accordance with this concept, includes at least one machine-readable non-transitory medium and information carried by the medium. The information carried by the medium may be executable program code data, parameters in association with the executable program code, and/or information related to a user, a request, content, or other additional information.

Another example is a machine-readable, non-transitory and tangible medium having information recorded thereon for robot-guided instrument insertion. A preplanned path between a three-dimensional (3D) entry pose on skin of a patient to a 3D pose of a target is generated for a surgery with a target inside the patient. The preplanned path as well as spatial relationship to surrounding anatomical structures are used by a robot as guidance to advance a surgical instrument from the 3D entry pose to reach the 3D pose of the target. During the surgery, a next pose is determined based on a current pose of the surgical instrument and the preplanned path and is used to move the surgical instrument thereto. An updated current pose of the instrument is then obtained via tracking when the robot is inserting the surgical instrument to the next pose. The process repeats until the instrument reaches the target pose.

Additional advantages and novel features will be set forth in part in the description which follows, and in part will become apparent to those skilled in the art upon examination of the following and the accompanying drawings or may be learned by production or operation of the examples. The advantages of the present teachings may be realized and attained by practice or use of various aspects of the methodologies, instrumentalities and combinations set forth in the detailed examples discussed below.

### BRIEF DESCRIPTION OF THE DRAWINGS

The methods, systems and/or programming described herein are further described in terms of exemplary embodiments. These exemplary embodiments are described in detail with reference to the drawings. These embodiments are non-limiting exemplary embodiments, in which like reference numerals represent similar structures throughout the several views of the drawings, and wherein:

FIG. 1 illustrates a pre-planned surgical path for a surgical instrument to reach a target yet avoid collision with other anatomical structures;

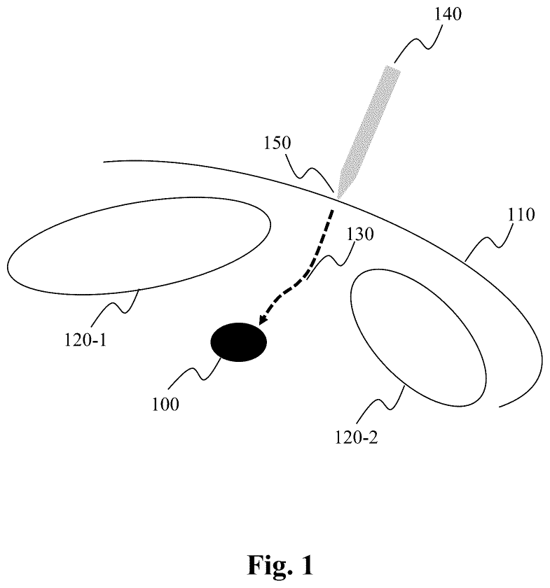

FIG. 2 depicts a surgical setting where a robot is deployed to manipulate a surgical instrument along a dynamically adjusted path to reach a target inside of a patient's body, in accordance with an embodiment of the present teaching;

FIG. 3A depicts an exemplary high-level system diagram of a framework for robot guided needle placement, in accordance with an embodiment of the present teaching;

FIG. 3B illustrates exemplary types of surgery related operational parameters;

FIG. 3C is a flowchart of an exemplary process of pre-surgical planning for a surgical needle insertion path, in accordance with an embodiment of the present teaching;

FIG. 3D is a flowchart of an exemplary process of during-surgery robot guided continuous needle placement to reach a target based on a preplanned surgical path, in accordance with an embodiment of the present teaching;

FIG. 4 depicts an exemplary high level system diagram of a next target needle pose determiner, in accordance with an embodiment of the present teaching;

FIG. 5A is a flowchart of an exemplary process for a next target needle pose determiner, in accordance with an embodiment of the present teaching;

FIG. 5B is a flowchart of an exemplary process for computing an adjusted needle pose under different situations, in accordance with an embodiment of the present teaching;

FIG. 6A illustrates an exemplary situation where a needle pose is dynamically adjusted to avoid an anticipated collision with another anatomical structure along a preplanned path, in accordance with an embodiment of the present teaching;

FIG. 6B illustrates an exemplary situation where an adjustment is made to a needle pose when there is no obstacle, in accordance with an embodiment of the present teaching;

FIG. 6C illustrates an exemplary situation where an adjustment is applied to a needle pose when the needle pose is close to an obstacle, in accordance with an embodiment of the present teaching;

FIG. 7 is an illustrative diagram of an exemplary mobile device architecture that may be used to realize a specialized system implementing the present teaching in accordance with various embodiments; and

FIG. 8 is an illustrative diagram of an exemplary computing device architecture that may be used to realize a specialized system implementing the present teaching in accordance with various embodiments.

### DETAILED DESCRIPTION

In the following detailed description, numerous specific details are set forth by way of examples in order to facilitate a thorough understanding of the relevant teachings. However, it should be apparent to those skilled in the art that the present teachings may be practiced without such details. In other instances, well known methods, procedures, components, and/or system have been described at a relatively high-level, without detail, in order to avoid unnecessarily obscuring aspects of the present teachings.

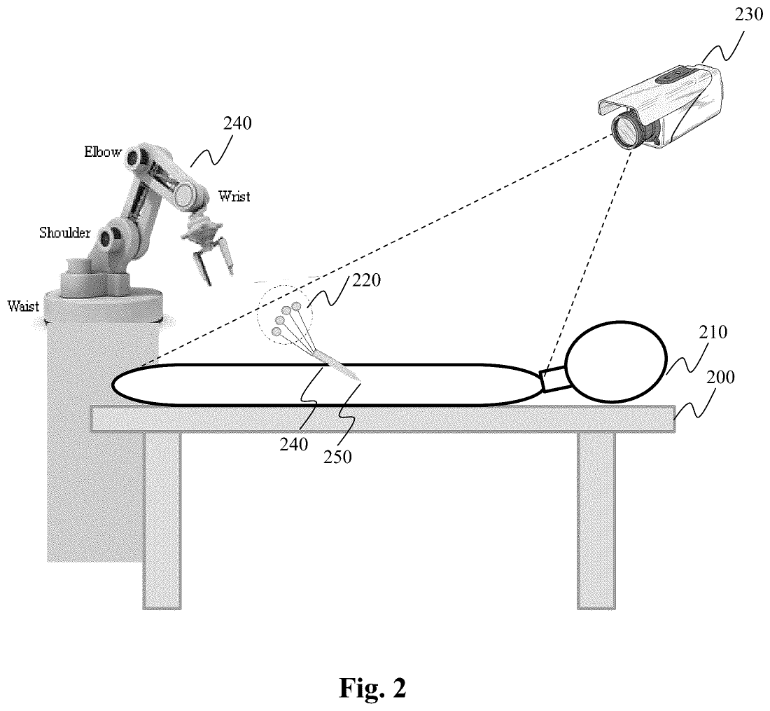

The present teaching discloses exemplary methods, systems, and implementations of a framework for robot guided surgical instrument insertion or placement. This is illustrated in a setting in FIG. 2, according to an embodiment of the present teaching. In this surgical setting, to perform a medical procedure on a patient 210 lying on a surgery table 200, a robot 240 may be deployed to manipulate the movement of a surgical instrument 240 with a tip 250 (such as a needle) to reach a target pose inside the patient's body. The 3D pose of the tip of the surgical instrument 240 may be obtained via a tracking mechanism, which includes a tracking camera 230 provided for monitoring the 3D pose of tracking element 220 within its field of view based on calibration that may map what is observed by camera 230 to 3D pose of the tracking element 220. In some embodiments, the 3D pose of the needle may be tracked through an electronic magnetic tracking system. Through the known spatial relation between tracking element 220 and the tip of the surgical instrument, the 3D pose of the tip of the surgical instrument may be accordingly determined. The robot 240 may operate to insert the surgical instrument 240 into the patient's body along a path determined based on a preplanned insertion path (generated prior to surgery) based on the observed poses of the surgical instrument as well as known positions of anatomical structures of the patient.

The framework of robot guided surgical instrument insertion according to the present teaching includes two parts, the pre-surgical planning part and an intra-operative part. The pre-surgical planning part is for planning a surgical instrument insertion path to reach a target inside of a patient (e.g., a lesion) based on 3D models of the patient's relevant anatomical structures. Such a preplanned insertion path generated prior to a surgery is then used by a robot in the intra-operative part of the framework to automatically insert a surgical instrument to reach the target with dynamic adjustments to the poses of the surgical instrument with respect to the preplanned surgical path under different situations. The robot may adjust the surgical instrument pose to deviate from the preplanned insertion path to avoid collisions or to keep a reasonable distance from anatomical structures nearby the target to ensure that the surgical instrument can safely reach the target without medical incidents.

According to the present teaching, during the insertion process, a situation associated with a current surgical instrument pose may be detected and the detected situation is used to determine a next target pose for the surgical instrument to reach. In a first situation, if the surgical instrument follows the preplanned insertion path, it will collide with an anatomical structure. This could be due to deformation of the anatomical structured of the patient so that even though the preplanned insertion path avoids such anatomical structured, during the surgery, this may change and require adjustment of the insertion path. In this first situation, a new next pose to be next reached by the surgical instrument may be computed that deviates from the preplanned insertion path in order to avoid the collision.

In a second situation, although the desired direction along the preplanned insertion path does not collide with any anatomical structure, the distance between the preplanner insertion path and some anatomical structure (e.g., an organ) may be too close. In this situation, the robot may adjust the surgical instrument's pose to deviate from the preplanned insertion path to increase the distance between surgical instrument and the nearby anatomical structure to ensure safety. In a third situation, when the surgical instrument is approaching the target (e.g., a lesion) without collision or being too close to other anatomical structure, the robot may still adjust the preplanned insertion path to ensure that the surgical instrument may smoothly approaching the target.

The present teaching may carry out a step-by-step adjustment process, in which it assesses the current instrument pose in relation to the preplanned insertion path to determine a next target pose for the surgical instrument to gradually approach the target (e.g., a lesion). With this approach, the preplanned insertion path may serve as a guide or a baseline but may be deviated with needed adjustments made based on actual situation observed during the surgery. This may effectively address the safety concern which may arise due to deformation of anatomical structures during the surgery.

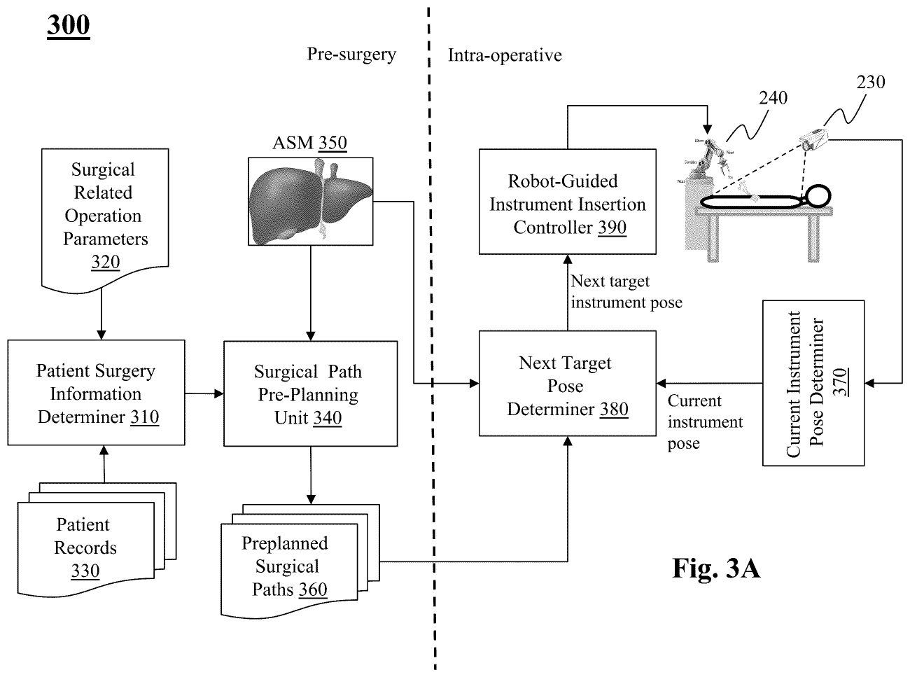

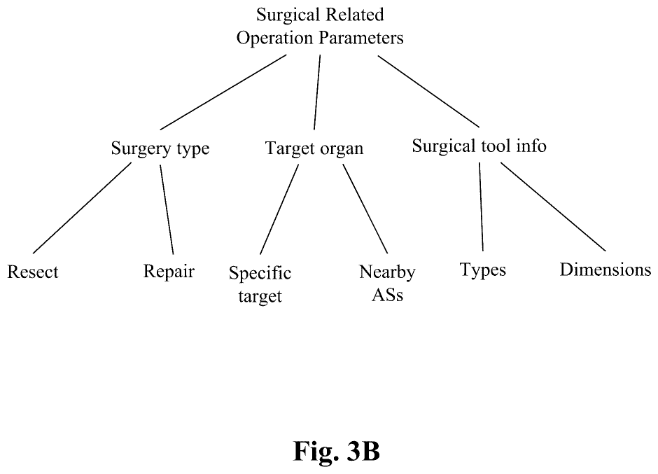

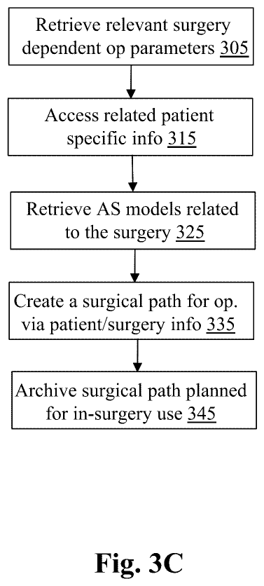

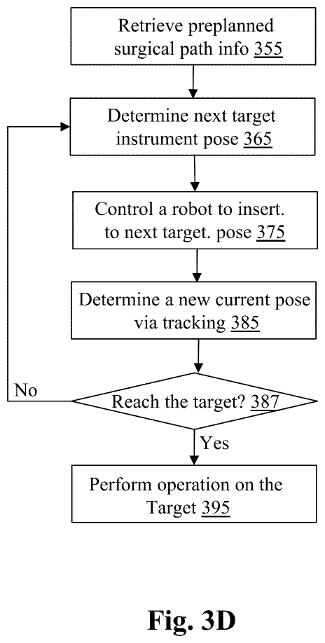

FIG. 3A depicts an exemplary high-level system diagram of the framework 300 for robot guided surgical instrument placement, in accordance with an embodiment of the present teaching. As stated herein, there are two parts, one corresponding to pre-surgery part for pre-surgical planning for generating preplanned surgical instrument insertion path and the other corresponding to intra-operative part where the preplanned surgical instrument insertion path is utilized by a robot to automatically insert a surgical instrument into a patient's body to reach a target pose (e.g., lesion to be removed). In this illustrated embodiment. The pre-surgery part comprises a patient surgery information determiner 310 and a surgical path pre-planning unit 340. The patient surgery information determiner 310 may be provided for obtaining information relevant to a patient (330) and an anticipated surgery (320). Such information may be relevant to the path to be preplanned to insert a surgical instrument.

FIG. 3B illustrates exemplary types of surgery related operational parameters, which may include, e.g., the type of surgery to be performed on a patient (e.g., to resect a lesion or to repair a damaged organ), the target organ (e.g., a lesion in the liver), . . . , and information about the surgical tool to be used to perform the procedure (e.g., a cutter to cut a lesion and a hook for move blood vessels away). Information related to a target organ may include both a specific target to be operated on (e.g., a lesion) and nearby anatomical structures (e.g., blood vessels and bones). Information about the surgical instrument to be inserted into the patient's body that is relevant to the generation of a preplanned insertion path may also be acquired, including the type(s) of surgical instrument as well as the dimension of each of the surgical instruments. Based on such relevant information, the surgical path pre-planning unit 340 may be provided to generate a preplanned surgical instrument insertion path (360) for the patient's medical procedure in accordance with anatomical structure models constructed for the patient (350).

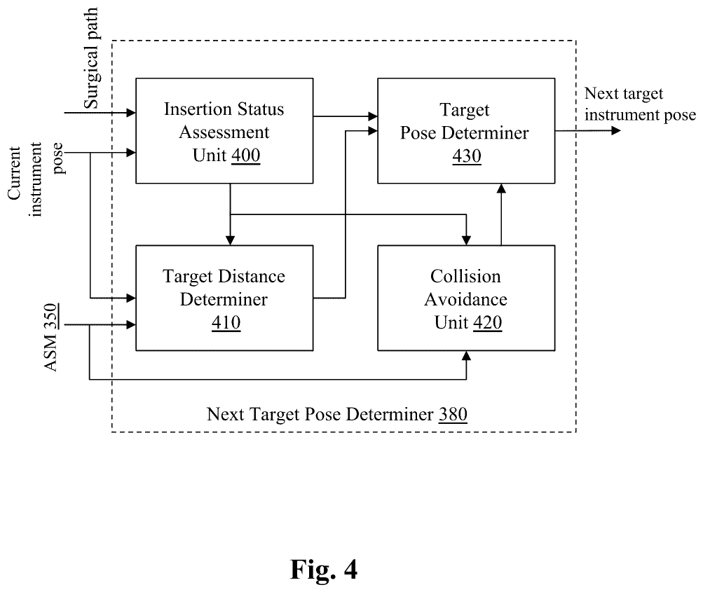

The intra-operative part of the framework 300 includes a current instrument pose determiner 370, a next target pose determiner 380, and a robot-guided instrument insertion controller 390. During the surgery, the next target pose determiner 380 is provided for accessing the preplanner surgical instrument insertion path 360 for the patient (generated prior to surgery) and uses that to guide the determination of the next target pose that the surgical instrument is to reach. As discussed herein, the preplanned insertion path may be used as a baseline which may be adjusted according to the real-time situation observed during the surgery. In some situations, the next target pose may correspond to a point along the preplanned insertion path. In some situation, the next target pose may correspond to an adjust pose that deviates from the preplanned insertion path for the safety of the patient during the surgery. Details related to adjustments to poses on the preplanned insertion path are provided with reference to FIGS. 4-6C.

The next target pose is then used by the robot-guided instrument insertion controller 390 to compute configuration parameters for the robot that are needed to move the surgical instrument to reach the next target pose. The current instrument pose determiner 370 is provided to determine the current pose of the surgical instrument, which may be based on the tracking mechanism deployed during the surgery. As shown in FIG. 2, the pose of the tip of the surgical instrument may be tracked via the tracking mechanism involving the tracking camera 230 as well as the tracking element 220. The tracked information may then be used by the current instrument pose determiner 370 to obtain the current pose of the tip of the surgical instrument. Based on the current pose of the surgical instrument, the next cycle starts again, i.e., the next target pose determiner 380 may determine the next target pose that the surgical instrument is to be moved to according to the present teaching.

FIG. 3C is a flowchart of an exemplary process of the first part of framework 300 for pre-surgical planning of a surgical instrument insertion path, in accordance with an embodiment of the present teaching. At 305, patient surgery information determiner 310 retrieves, from the surgical related operation parameter storage 320, relevant surgery dependent operation parameters and accesses, at 315 from the patient records 330, related patient specific information such as, e.g., patient's age, diagnosis, surgery preparation condition, etc. Such relevant information is then provided to the surgical path pre-planning unit 340, which then accordingly retrieves, at 325, three-dimensional models for the target organ as well as relevant anatomical structures that are either near the target organ or between the target organ and the skin of the patient. The retrieved 3D models may then be used for the surgical path pre-planning unit 340 to create, at 335, a surgical instrument insertion path for the operation. Such a created surgical insertion path may then be archived, at 345, in the preplanned surgical path storage 360, which may be retrieved during the surgery.

FIG. 3D is a flowchart of an exemplary process of the second part of framework 300 for intra-operative robot guided continuous surgical instrument placement to reach a target based on a preplanned surgical instrument insertion path, in accordance with an embodiment of the present teaching. When the surgery for a patient is initiated, the next target pose determiner 380 retrieves, at 355, a preplanned surgical instrument insertion path from 360, and then determines, at 365, a next target instrument pose for the surgical instrument to be reached next. The robot-guided instrument insertion controller 390 then computes, based on the next target pose, the robot arm configuration parameters needed to facilitate the robot 240 to manipulate the surgical instrument to move to the next target instrument pose at 375. The movement may then be tracked, and the new current instrument pose is obtained, at 385, by the current instrument pose determiner 370. If the current instrument pose reached the target, determined at 387, the insertion of the surgical instrument is completed so that the operation may be performed at 395. If the current instrument pose is not yet at the target, the processing moves to step 365 to start the next cycle. The iterative process continues until the instrument reaches the target.

FIG. 4 depicts an exemplary high-level system diagram of the next target pose determiner 380, in accordance with an embodiment of the present teaching. As discussed herein, the next target pose determiner 380 is invoked when the current instrument pose is not yet at the designated target so that a next target pose for the instrument to approach is determined. As shown in FIG. 4, the next target pose determiner 380 takes the current instrument pose, the preplanned surgical instrument insertion path, as well as the ASM models 350 as input and determines the next target pose accordingly. In this illustrated embodiment, the next target pose determiner 380 comprises an insertion status assessment unit 400, a target distance determiner 410, a collision assessment unit 420, and a target pose determiner 430. The insertion status assessment unit 400 is provided for evaluating the relationship between a current instrument pose and the preplanned instrument insertion path, e.g., whether the current instrument pose is on or deviating from the preplanned insertion path.

The target distance determiner 410 may be provided for determining a situation the current instrument pose is in with respect to, e.g., the target organ or other anatomical structures. For example, given a 3D pose of the surgical instrument, it may be evaluated as to whether the instrument is to collide with some anatomical structure (e.g., an organ) if the surgical instrument continues the current insertion direction. It may also be evaluated as to the distance between the current instrument pose and nearby anatomical structures. Such an assessment may be performed with respect to the 3D models for the anatomical structures, e.g., based on the current instrument pose, its distance to nearby anatomical structures may be determined represented by the 3D anatomical structure models. In addition, the collision assessment unit 420 may be provided to evaluate as to whether a collision may occur with any of the anatomical structures. Based on the evaluation on which specific situation is associated with the current instrument pose, the target pose determiner 430 may then accordingly compute the next target pose to be reached by the surgical instrument in the current iteration.

As discussed herein, although a preplanned insertion path is provided, during a surgery, due to different reasons, the actual insertion path may deviate from the preplanned insertion path due to, e.g., displacement of different anatomical structures during the surgery and/or deformation of some anatomical structures. In each iteration, a situation associated with a current surgical instrument pose may be determined so that the next target instrument pose may be determined accordingly. In some situations, the next target instrument pose (which corresponds to a next insertion direction from the current instrument pose) may be determined according to the preplanned insertion path. In some situations, the next insertion pose specified in the preplanned insertion path may not be adopted and instead may deviate from what is preplanned to avoid problems or improve safety.

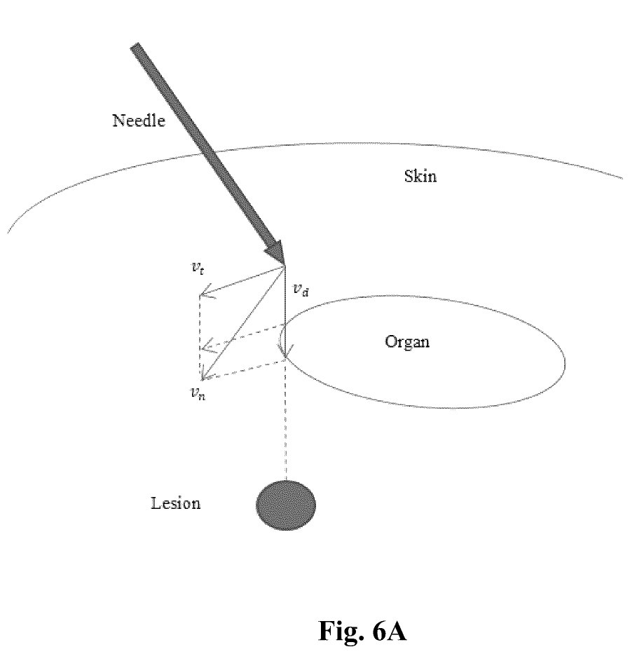

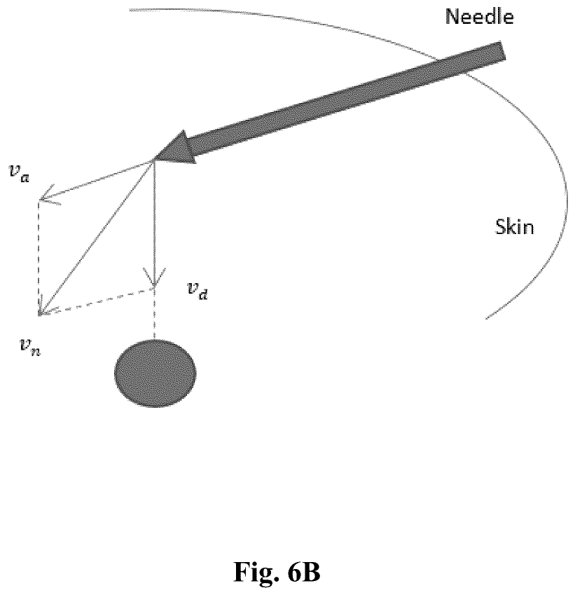

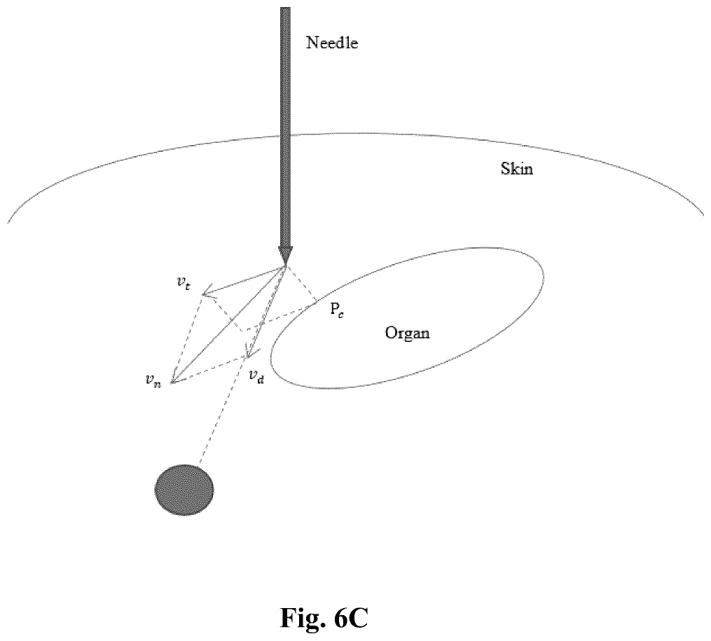

FIGS. 6A-6C show different situations that lead to different computations of the next target instrument pose with illustration of a surgical needle as a surgical instrument and a lesion as a target. In these illustrations, v_{d} denotes a desired orientation vector representing the next insertion direction from a current needle tip according to a preplanned insertion path. FIG. 6A shows a situation in which the desired insertion direction v_{d} extending from the current needle tip according to a preplanned insertion path will causes, if adopted, a collision with an obstructing organ. As such, in this situation, the desired direction from the preplanned insertion path may not be adopted as it will cause a collision and instead an alternative next target instrument pose may be determined to deviate from the preplanned insertion path to avoid the collision. This collision situation may occur when some anatomical structures of the patient may be displaced during the surgery (e.g., due to how the patient is lying on the surgery bed) or some may have deformed during the surgery.

FIGS. 6B-6C show additional scenarios where between the desired insertion orientation v_{d}, extended from the needle tip along the preplanned insertion path and the target lesion, there is no collision. FIG. 6B involves a situation where the needle tip is sufficiently away from any other anatomical structures, while FIG. 6C shows a situation where the needle tip is sufficiently nearby another organ even though the desired insertion direction v_{d} is not obstructed by the organ. In the situation illustrated in FIG. 6B, the desired insertion direction v_{d} according to the preplanned insertion path may be adopted to control the next needle movement according to the preplanned insertion path as it is not going to cause any safety concerns. However, in the situation illustrated in FIG. 6C, due to the close distance between the needle tip and another organ, to enhance safety, the desired insertion direction v_{d} may not be adopted and the next target instrument pose may be computed to deviate from the preplanned insertion path to minimize the risks.

Below, detailed computation of the next target instrument pose as determined based on different situations is discussed. In operation, when the surgical instrument is inserted into a patient through, e.g., a tool guide deployed on the robot, a reading from a sensor on the tip of the surgical instrument (e.g., a needle) may be obtained via the tracking mechanism as discussed herein. Denote the current instrument pose (3D position and 3D orientation) as Pₙ=(xₙ, yₙ, zₙ, aₙ, eₙ, rₙ), where pₐ=(xₙ, yₙ, zₙ) is current position of the instrument tip and vₐ=(aₙ, eₙ, rₙ) is the current orientation unit vector of the instrument, where a, e, and r may represent pitch, roll, and yaw, respectively. Further assume that the preplanned position for the instrument tip is p_{d}(x_{d}, y_{d}, z_{d}) and preplanned orientation vector of the instrument is v_{d}=(a_{d}, e_{d}, r_{d}) based on the current instrument position. As discussed herein, the desired (preplanned) instrument position p_{d} and orientation v_{d} may be a continuous function or discrete samples arranged in a sequence. For example, a trajectory to reach the desired instrument positions p_{d} may be a list of discrete points starting from an entry point on the skin to the target (e.g., lesion center). In some embodiments, the trajectory of a series of desired preplanned instrument orientation v_{d} may be represented as a collection of vectors starting from the entry point to the target. In some embodiments, the pose trajectory of the preplanned insert path may be a smoothed version of an initial desired pose trajectory. Such smoothing may be introduced to reduce, e.g., sharp angles formed by adjacent poses for the purpose of, e.g., enabling more accurate tracking during the surgery.

In determining the next target instrument pose, in each iteration after the instrument is already inserted into the skin, it may be checked whether the next target instrument pose needs to deviate from the desired trajectory according to the situation detected. If the current instrument position is deviated from the preplanned insertion path, the next target instrument position may be calculated as:

pₙ=p_{d}

and the next target instrument orientation vector may be calculated as

$${v}_{n} = {v}_{a} + s ( {d}_{s} ) * {v}_{d}$$

where s(d) is a function of distance dₛ between the instrument tip position pₙ and the target for the insertion (e.g., a lesion center). See FIG. 6B. The function s(dₛ) may be provided to have a larger value if dₛ is smaller. vₐ corresponds to the current orientation of the surgical instrument.

At each iteration at an instrument pose, when vector vₙ₋₁ of the surgical instrument at step n−1 is in collision with any anatomical structure (such as an organ, as illustrated in FIG. 6A) or if the minimum distance dᵥ between vector vₙ₋₁ and a closest non-target anatomical structure is below a preset threshold (as shown in FIG. 6C), the next target instrument orientation may be calculated as

$${v}_{n} = {v}_{d} + f ( {d}_{0} ) * {v}_{t}$$

where vₜ is, in situation shown in FIG. 6A, a tangent vector along a direction of the surface of the anatomical structure at the cross point that the desired needle vector v_{d} collides with the anatomical structure. When vector vₙ at the instrument tip does not pass through the organ (situation in FIG. 6C), vₜ is a tangent vector along a direction at the closest point Pc on the surface of the anatomical structure from the tip of the surgical instrument. In some embodiments, f(dₒ) may correspond to a function of the minimum distance dₒ between the position pₙ of the tip of the surgical instrument and the anatomical structure. In some embodiments, the size of the anatomical structure that is obstructing the desired needle vector v_{d} as well as the how much the surgical instrument needs to be bended to reach the next target instrument pose may also be considered and incorporated into the equation to determine if the needle path can avoid the organ collision.

As shown above, at each current pose during an insertion process, if a surgical instrument has not yet reached the target, then a next target instrument pose may be determined as the target location the tip of the surgical instrument is to reach in the next iteration and is computed based on a specific situation detected to avoid potential collision or enhance the operability of the insertion. The present teaching is capable of deviating, when needed, the preplanner insertion path based on dynamically detected situation to adjust the next target instrument pose to a location that is safe for the patient. Once a next target instrument pose is determined, to move the surgical instrument to the next target instrument pose, the robot joint position may then be calculated to effectuate the manipulation of the surgical instrument towards the next target instrument pose. It must be appreciated that the pose of the surgical instrument relative to the robot base is known through certain calibration. The iterations may continue until the tip of the surgical instrument is sufficiently close to the target.

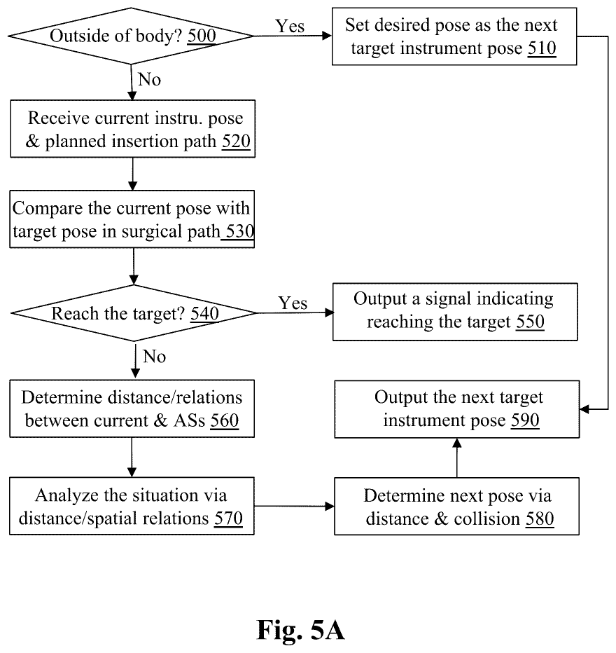

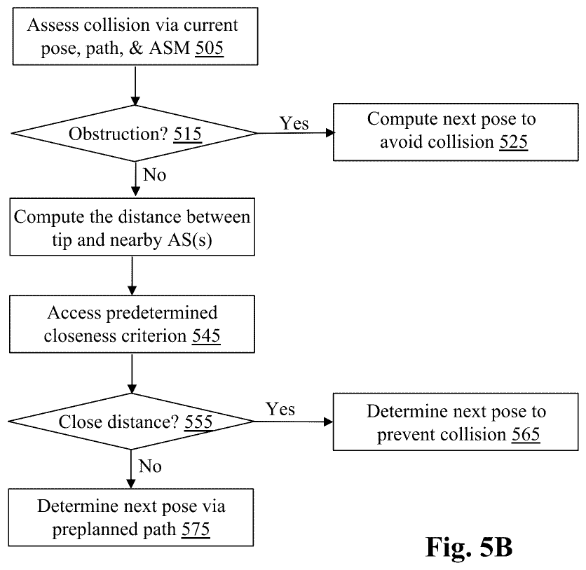

FIG. 5A is a flowchart of an exemplary process for the next target pose determiner 380, in accordance with an embodiment of the present teaching. To compute the next target instrument pose, it is first checked, at 500, whether the pose of the surgical instrument to be inserted is currently outside of the patient's body. If it is outside of the patient's body, the next target instrument pose is set, at 510, based on the desired pose of the target and then output, at 590, the set next target instrument pose. When the surgical instrument is already inside of the patient's body, to compute the next target instrument pose, various information to be used in determining the next target instrument pose is received, at 520, by the next target pose determiner 380, which includes the current pose of the surgical instrument, the preplanned insertion path, as well as ASM 350. Based on the received information, the current pose of the surgical instrument is compared, at 530, with the pose of the target (e.g., lesion center) specified in the preplanned insertion path.

If the surgical instrument has reached the target, determined at 540, then the insertion process is completed and a signal is output, at 550, to indicate that the surgical instrument has reached the target. If the surgical instrument has not yet reached the target, the distance and spatial relations between the current pose of the surgical instrument and target and other nearby relevant anatomical structures are determined at 560. The situation the current instrument pose is in is analyzed at 570. Based on such determined distance, spatial relations, and the detected situation, the next targe instrument pose is then computed at 580 in accordance with the present teaching (e.g., the formulations as illustrated herein) with respect to the detected situation. The determined next target instrument pose is then output at 590.

FIG. 5B is a flowchart of an exemplary process for exemplary steps of determining the next target instrument pose under different situations, in accordance with an embodiment of the present teaching. At 505, whether there will be a collision is first assessed based on the current instrument pose, the preplanned insertion path, and 3D models for anatomical structures near the current instrument pose. If obstruction is detected (which is the situation depicted in FIG. 6A), determined at 515, the desired insertion pose from the preplanned insertion path is not adopted and instead a target instrument pose is computed, at 525, according to the above formulation to avoid the detected collision. If no anticipated collision is detected (which is a situation of either FIG. 6B or FIG. 6C), the distance between the instrument tip and nearby anatomical structures, if any, is computed at 535 and will be used to further assess whether the current situation is what is depicted in FIG. 6B or in FIG. 6C.

To use the computed distance to determine the specific situation, a predetermined criterion defining what constitute close distance is accessed at 545 and then used to determine, at 555, whether the computed distance meets the criterion for being close to an anatomical structure. If the closeness condition is not satisfied (a situation as depicted in FIG. 6B), then the next insertion pose as specified in the preplanned insertion path may be adopted as the next target instrument pose. Otherwise, the tip of the instrument may be considered too close to an obstruction (as shown in FIG. 6C) so that an alternative pose for the next move is computed at 565 in accordance with the formulation provided herein for this situation. This process produces a next target instrument pose which is either adopted from the preplanned insertion path or generated as an adjusted next target instrument pose that deviates from the pose specified in the preplanned insertion path according to the specific situation detected. As discussed herein, the adaptively computed next target instrument pose is then used to adjust the robot joint position so to facilitate robot arm actions to move the surgical instrument to the next target instrument pose. As the relative spatial relationship between the pose of the tip of the instrument and that of the robot base is known through calibration, the adjustment to the robot joint position needed is a matter of kinematic transformation.

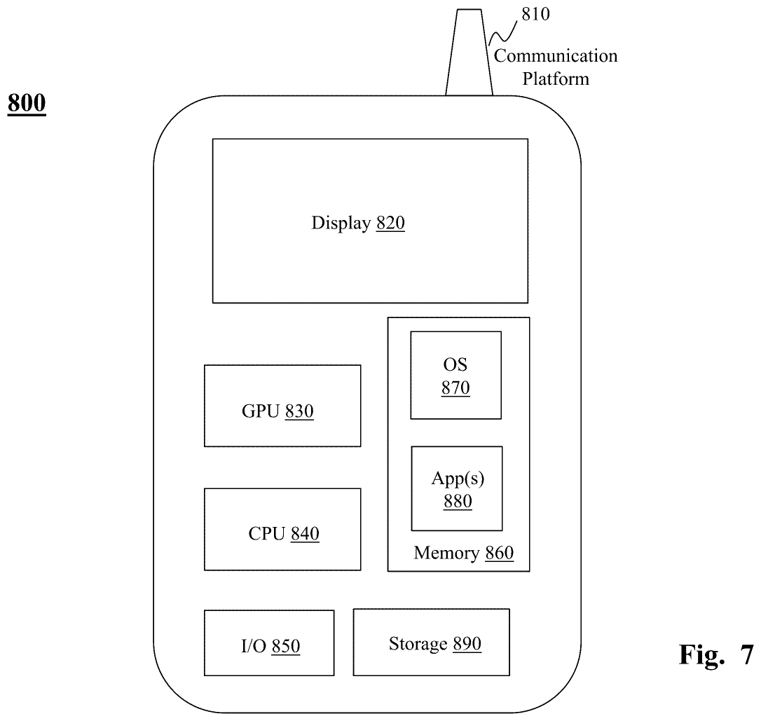

FIG. 7 is an illustrative diagram of an exemplary mobile device architecture that may be used to realize a specialized system implementing the present teaching in accordance with various embodiments. In this example, the user device on which the present teaching may be implemented corresponds to a mobile device 700, including, but not limited to, a smart phone, a tablet, a music player, a handled gaming console, a global positioning system (GPS) receiver, and a wearable computing device, or in any other form factor. Mobile device 700 may include one or more central processing units (“CPUs”) 740, one or more graphic processing units (“GPUs”) 730, a display 720, a memory 760, a communication platform 710, such as a wireless communication module, storage 790, and one or more input/output (I/O) devices 750. Any other suitable component, including but not limited to a system bus or a controller (not shown), may also be included in the mobile device 700. As shown in FIG. 7, a mobile operating system 770 (e.g., iOS, Android, Windows Phone, etc.), and one or more applications 780 may be loaded into memory 760 from storage 790 to be executed by the CPU 740. The applications 780 may include a user interface or any other suitable mobile apps for information analytics and management according to the present teaching on, at least partially, the mobile device 700. User interactions, if any, may be achieved via the I/O devices 750 and provided to the various components connected via network(s).

To implement various modules, units, and their functionalities described in the present disclosure, computer hardware platforms may be used as the hardware platform(s) for one or more of the elements described herein. The hardware elements, operating systems and programming languages of such computers are conventional in nature, and it is presumed that those skilled in the art are adequately familiar with to adapt those technologies to appropriate settings as described herein. A computer with user interface elements may be used to implement a personal computer (PC) or other type of workstation or terminal device, although a computer may also act as a server if appropriately programmed. It is believed that those skilled in the art are familiar with the structure, programming, and general operation of such computer equipment and as a result the drawings should be self-explanatory.

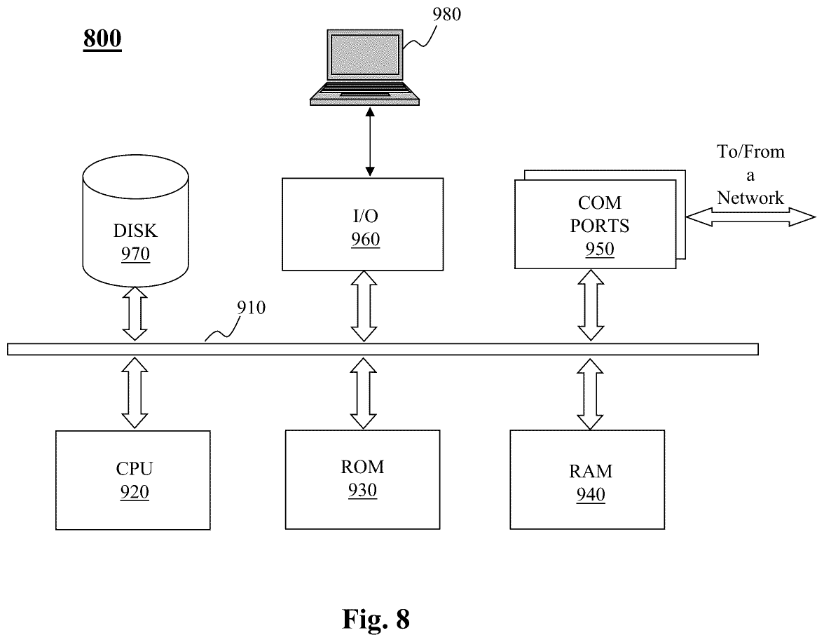

FIG. 8 is an illustrative diagram of an exemplary computing device architecture that may be used to realize a specialized system implementing the present teaching in accordance with various embodiments. Such a specialized system incorporating the present teaching has a functional block diagram illustration of a hardware platform, which includes user interface elements. The computer may be a general-purpose computer or a special purpose computer. Both can be used to implement a specialized system for the present teaching. This computer 800 may be used to implement any component or aspect of the framework as disclosed herein. For example, the information analytical and management method and system as disclosed herein may be implemented on a computer such as computer 800, via its hardware, software program, firmware, or a combination thereof. Although only one such computer is shown, for convenience, the computer functions relating to the present teaching as described herein may be implemented in a distributed fashion on a number of similar platforms, to distribute the processing load.

Computer 800, for example, includes COM ports 850 connected to and from a network connected thereto to facilitate data communications. Computer 800 also includes a central processing unit (CPU) 820, in the form of one or more processors, for executing program instructions. The exemplary computer platform includes an internal communication bus 810, program storage and data storage of different forms (e.g., disk 870, read only memory (ROM) 830, or random-access memory (RAM) 840), for various data files to be processed and/or communicated by computer 800, as well as possibly program instructions to be executed by CPU 820. Computer 800 also includes an I/O component 860, supporting input/output flows between the computer and other components therein such as user interface elements 880. Computer 800 may also receive programming and data via network communications.

Hence, aspects of the methods of information analytics and management and/or other processes, as outlined above, may be embodied in programming. Program aspects of the technology may be thought of as “products” or “articles of manufacture” typically in the form of executable code and/or associated data that is carried on or embodied in a type of machine-readable medium. Tangible non-transitory “storage” type media include any or all of the memory or other storage for the computers, processors or the like, or associated modules thereof, such as various semiconductor memories, tape drives, disk drives and the like, which may provide storage at any time for the software programming.

All or portions of the software may at times be communicated through a network such as the Internet or various other telecommunication networks. Such communications, for example, may enable loading of the software from one computer or processor into another, for example, in connection with information analytics and management. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links, or the like, also may be considered as media bearing the software. As used herein, unless restricted to tangible “storage” media, terms such as computer or machine “readable medium” refer to any medium that participates in providing instructions to a processor for execution.

Hence, a machine-readable medium may take many forms, including but not limited to, a tangible storage medium, a carrier wave medium or physical transmission medium. Non-volatile storage media include, for example, optical or magnetic disks, such as any of the storage devices in any computer(s) or the like, which may be used to implement the system or any of its components as shown in the drawings. Volatile storage media include dynamic memory, such as a main memory of such a computer platform. Tangible transmission media include coaxial cables; copper wire and fiber optics, including the wires that form a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a physical processor for execution.

Those skilled in the art will recognize that the present teachings are amenable to a variety of modifications and/or enhancements. For example, although the implementation of various components described above may be embodied in a hardware device, it may also be implemented as a software only solution, e.g., an installation on an existing server. In addition, the techniques as disclosed herein may be implemented as a firmware, firmware/software combination, firmware/hardware combination, or a hardware/firmware/software combination.

While the foregoing has described what are considered to constitute the present teachings and/or other examples, it is understood that various modifications may be made thereto and that the subject matter disclosed herein may be implemented in various forms and examples, and that the teachings may be applied in numerous applications, only some of which have been described herein. It is intended by the following claims to claim any and all applications, modifications and variations that fall within the true scope of the present teachings.

We claim:

1. A method implemented on at least one processor, a memory, and a communication platform, comprising:
retrieving a preplanned path generated for a surgery on a patient with respect to a target inside the patient, wherein the preplanned path is between a three dimensional (3D) entry pose on skin of the patient to a 3D pose of the target and provided to a robot to insert a surgical instrument from the 3D entry pose to reach the 3D pose of the target;
determining a next pose for the surgical instrument based on a current pose of the surgical instrument and the preplanned path as well as spatial relationship to surrounding anatomical structures;
controlling the robot to move the surgical instrument to reach the next pose;
obtaining an updated current pose of the surgical instrument via tracking when the robot is advancing the surgical instrument to the next pose;
repeating the steps of determining, controlling, and obtaining if the updated current pose is not the 3D pose of the target determined based on a predetermined criterion; and
outputting a signal indicating that the surgical instrument reaches the 3D pose of the target when the updated current pose reaches the 3D pose of the target based on the predetermined criterion.

2. The method of claim 1, wherein the step of determining the next pose comprises:
retrieving 3D models constructed to characterize anatomical structures of the patient;
deriving, based on the 3D models, spatial relationships between the current pose of the surgical instrument and at least some of the anatomical structures; and
computing the next pose based on the spatial relationships and the 3D pose of the target in the preplanned path, wherein
the spatial relationships include a distance to and a spatial configuration with respect to each of the at least some of the anatomical structures.

3. The method of claim 2, wherein the step of computing the next pose comprises:
retrieving a subsequent pose on the preplanned path as a candidate next pose;
adopting the candidate next pose as the next pose if the candidate next pose does not cause collision or present a risk of collision with any of the anatomical structures; and
modifying the candidate next pose to derive the next pose to avoid a collision or prevent a risk of collision with any of the anatomical structures.

4. The method of claim 3, wherein
the collision is determined based on an orientation associated with the candidate next pose; and
the risk of collision with respect to an anatomical structure is determined based on a distance between the candidate next pose and the anatomical structure.

5. The method of claim 1, wherein the step of controlling the robot comprises:
determining differences in a first set of parameters used to configure the robot to move the surgical instrument to the current pose and a second set of parameters needed to configure the robot to move the surgical instrument to the next pose; and
configuring the robot based on the differences to enable the robot to control the movement of the surgical instrument from the current pose to the next pose.

6. The method of claim 1, wherein the predetermined criterion is defined in accordance with a distance between the current pose and the 3D pose of the target.

7. The method of claim 1, wherein the preplanned path is generated by:
obtaining
information related to the patient, and
operational parameters associated with the surgery;
retrieving the 3D models constructed for characterizing the anatomical structures of the patient;
estimating a starting 3D pose for the entry on the skin of the patient and an ending 3D pose of the target;
determining a plurality of 3D poses between the starting 3D pose and the ending 3D pose without collision with the anatomical structures according to the 3D models; and
creating the preplanned path based on the plurality of 3D poses.

8. Machine readable and non-transitory medium having information recorded thereon, wherein the information, when read by the machine, causes the machine to perform the following steps:
retrieving a preplanned path generated for a surgery on a patient with respect to a target inside the patient, wherein the preplanned path is between a three dimensional (3D) entry pose on skin of the patient to a 3D pose of the target and provided to a robot to insert a surgical instrument from the 3D entry pose to reach the 3D pose of the target;
determining a next pose for the surgical instrument based on a current pose of the surgical instrument and the preplanned path as well as spatial relationship to surrounding anatomical structures;
controlling the robot to move the surgical instrument to reach the next pose;
obtaining an updated current pose of the surgical instrument via tracking when the robot is advancing the surgical instrument to the next pose;
repeating the steps of determining, controlling, and obtaining if the updated current pose is not the 3D pose of the target determined based on a predetermined criterion; and
outputting a signal indicating that the surgical instrument reaches the 3D pose of the target when the updated current pose reaches the 3D pose of the target based on the predetermined criterion.

9. The medium of claim 8, wherein the step of determining the next pose comprises:
retrieving 3D models constructed to characterize anatomical structures of the patient;
deriving, based on the 3D models, spatial relationships between the current pose of the surgical instrument and at least some of the anatomical structures; and
computing the next pose based on the spatial relationships and the 3D pose of the target in the preplanned path, wherein
the spatial relationships include a distance to and a spatial configuration with respect to each of the at least some of the anatomical structures.

10. The medium of claim 9, wherein the step of computing the next pose comprises:
retrieving a subsequent pose on the preplanned path as a candidate next pose;
adopting the candidate next pose as the next pose if the candidate next pose does not cause collision or present a risk of collision with any of the anatomical structures; and
modifying the candidate next pose to derive the next pose to avoid a collision or prevent a risk of collision with any of the anatomical structures.

11. The medium of claim 10, wherein
the collision is determined based on an orientation associated with the candidate next pose; and
the risk of collision with respect to an anatomical structure is determined based on a distance between the candidate next pose and the anatomical structure.

12. The medium of claim 8, wherein the step of controlling the robot comprises:
determining differences in a first set of parameters used to configure the robot to move the surgical instrument to the current pose and a second set of parameters needed to configure the robot to move the surgical instrument to the next pose; and
configuring the robot based on the differences to enable the robot to control the movement of the surgical instrument from the current pose to the next pose.

13. The medium of claim 8, wherein the predetermined criterion is defined in accordance with a distance between the current pose and the 3D pose of the target.

14. The medium of claim 8, wherein the preplanned path is generated by:
obtaining
information related to the patient, and
operational parameters associated with the surgery;
retrieving the 3D models constructed for characterizing the anatomical structures of the patient;
estimating a starting 3D pose for the entry on the skin of the patient and an ending 3D pose of the target;
determining a plurality of 3D poses between the starting 3D pose and the ending 3D pose without collision with the anatomical structures according to the 3D models; and
creating the preplanned path based on the plurality of 3D poses.

15. A system, comprising:
a next pose determiner implemented by a processor and configured for
retrieving a preplanned path generated for a surgery on a patient with respect to a target inside the patient, wherein the preplanned path is between a three dimensional (3D) entry pose on skin of the patient to a 3D pose of the target and provided to a robot to insert a surgical instrument from the 3D entry pose to reach the 3D pose of the target, and
determining a next pose for the surgical instrument based on a current pose of the surgical instrument and the preplanned path as well as spatial relationship to surrounding anatomical structures;
a robot-guided instrument insertion controller implemented by a processor and configured for controlling the robot to move the surgical instrument to reach the next pose; and
a current instrument pose determiner implemented by a processor and configured for obtaining an updated current pose of the surgical instrument via tracking when the robot is advancing the surgical instrument to the next pose, wherein
the next pose determiner, the robot-guided instrument insertion controller, and the current instrument pose determiner are configured for
repeating the steps of determining, controlling, and obtaining if the updated current pose is not the 3D pose of the target determined based on a predetermined criterion, and
outputting a signal indicating that the surgical instrument reaches the 3D pose of the target when the updated current pose reaches the 3D pose of the target based on the predetermined criterion.

16. The system of claim 15, wherein the next pose determiner is configured for determining the next pose by:
retrieving 3D models constructed to characterize anatomical structures of the patient;
deriving, based on the 3D models, spatial relationships between the current pose of the surgical instrument and at least some of the anatomical structures; and
computing the next pose based on the spatial relationships and the 3D pose of the target in the preplanned path, wherein
the spatial relationships include a distance to and a spatial configuration with respect to each of the at least some of the anatomical structures.

17. The system of claim 16, wherein the step of computing the next pose comprises:
retrieving a subsequent pose on the preplanned path as a candidate next pose;
adopting the candidate next pose as the next pose if the candidate next pose does not cause collision or present a risk of collision with any of the anatomical structures; and
modifying the candidate next pose to derive the next pose to avoid a collision or prevent a risk of collision with any of the anatomical structures.

18. The system of claim 17, wherein
the collision is determined based on an orientation associated with the candidate next pose; and
the risk of collision with respect to an anatomical structure is determined based on a distance between the candidate next pose and the anatomical structure.

19. The system of claim 15, wherein the robot-guided instrument insertion controller is configured for controlling the robot by:
determining differences in a first set of parameters used to configure the robot to move the surgical instrument to the current pose and a second set of parameters needed to configure the robot to move the surgical instrument to the next pose; and
configuring the robot based on the differences to enable the robot to control the movement of the surgical instrument from the current pose to the next pose.

20. The system of claim 15, wherein the predetermined criterion is defined in accordance with a distance between the current pose and the 3D pose of the target.

21. The system of claim 15, further comprising a surgical path preplanning unit implemented by a processor and configured for generating the preplanned path by:
obtaining
information related to the patient, and
operational parameters associated with the surgery;
retrieving the 3D models constructed for characterizing the anatomical structures of the patient;
estimating a starting 3D pose for the entry on the skin of the patient and an ending 3D pose of the target;
determining a plurality of 3D poses between the starting 3D pose and the ending 3D pose without collision with the anatomical structures according to the 3D models; and
creating the preplanned path based on the plurality of 3D poses.
